(19) United States
(12) Patent Application Publication
(10) Pub. No.: US 2005/0112154 A1
(43) Pub. Date: May 26, 2005

(54) Cosmetic composition comprising particles having a core-shell structure

(76) Inventors: Franck Giroud, Clichy (FR); Henri Samain, Bievres (FR); Isabelle Rollat, Paris (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/915,432

(22) Filed: Aug. 11, 2004

Related U.S. Application Data
(60) Provisional application No. 60/511,579, filed on Oct. 16, 2003.

(30) Foreign Application Priority Data
Aug. 11, 2003 (FR) 03 50419

(51) Int. Cl. A61K 7/42; A61K 7/06; A61K 7/11

(52) U.S. Cl. 424/401; 424/59; 424/70.13; 424/70.14

(57) ABSTRACT

Disclosed herein is a cosmetic composition comprising, in a physiologically acceptable medium, at least one agent exhibiting a cosmetic activity and particles comprising a core and a solid shell bonded to the core via a noncovalent bond, wherein the core predominantly comprises at least one metal, the solid shell comprises at least one organic material, and the size of the particles is less than or equal to 500 nm. Further disclosed herein is a cosmetic process for the treatment of keratinous substances, such as hair, for example, for contributing sheen to the keratinous substances, comprising applying to the keratinous substances the composition, as well as the use of the core-shell particles in a cosmetic composition for contributing sheen to the keratinous substances.

This application claims benefit of U.S. Provisional Application No. 60/511,579 filed Oct. 16, 2003.

Disclosed herein is a cosmetic composition comprising particles having a core-shell structure.

Further disclosed herein is a cosmetic process for the treatment of keratinous substances, such as hair, for example, for contributing sheen thereto, using the disclosed composition.

Even further disclosed herein is the use of the composition for contributing sheen to keratinous substances such as the hair.

The technical field of the invention can be defined as that of cosmetic compositions, such as hair compositions and compositions for skin or nails.

The use of metal particles has already been disclosed in various types of cosmetic make-up compositions.

The document EP-A-1 082 952 discloses make-up compositions, such as for the nails, comprising glass particles covered with a metal layer which make it possible to obtain a make-up exhibiting a sparkling and wear-resistant metallic appearance.

The document EP-A-953 330 relates to the combination of two different compositions respectively comprising metal particles of goniochromatic pigment type and a pigment of conventional type having one of the colors of the first pigment for producing a make-up with a metallic effect which can vary according to the angle of observation and which can exhibit iridescent effects.

More recently, International Patent Application WO-A-02/03913 disclosed nail varnish compositions comprising particles in the form of aluminium platelets in proportions by weight of 0.4% to 0.75% and film-forming agents having high molecular weights for producing a make-up of mirror type, that is to say, in this instance, a make-up having not only the color of the aluminium but also a sheen and an ability to reflect the separate components of an object.

Metal particles have also been incorporated in hair compositions. For example, it is possible to contribute to the hair a better sheen than that contributed by fatty substances by incorporating metal nanoparticles, such as silver nanoparticles, in hair compositions. Such compositions are disclosed in the document EP-A-1 064-918; however, it has been found that the sheen contributed by such compositions may fade very rapidly over time.

In another field, the document WO-A-00/78282 discloses the use of silver nanoparticles with a size of 1 nm to 50 nm as antimicrobial agent in curable silicone rubber compositions. This document, however, does not disclose the use of encapsulated nanoparticles.

There thus exists a still unfulfilled need for a cosmetic composition, such as a hair cosmetic composition, comprising metal particles, which can have a high sheen, wherein the sheen can be maintained over a long period of time and wherein there is minimal to no fading over time.

There also exists a need for a cosmetic composition, such as a hair composition, which, while exhibiting a high sheen and over a long period of time, may also have great stability over time.

The present disclosure provides a cosmetic composition which meets, inter alia, at least one of these needs.

The present disclosure also provides a cosmetic composition which may not exhibit the disadvantages, failings, limitations and inconveniences of the compositions of the prior art and which can solve at least one of the problems of the compositions of the prior art.

Disclosed herein, therefore, is a cosmetic composition comprising, in a physiologically acceptable medium, at least one agent exhibiting a cosmetic activity and particles comprising a core and a solid shell bonded to the core via a noncovalent bond, wherein the core predominantly comprises at least one metal, the solid shell comprises at least one organic material, and the size of the particles is less than or equal to 500 nm.

Cosmetic compositions as described above comprising the specific particles incorporated in the compositions disclosed herein, which are defined by a specific structure, specific constituents and a specific particle size, have never been mentioned in the prior art.

Surprisingly, as a result of the incorporation in the compositions disclosed herein of these specific particles, which may be described as encapsulated metal nanoparticles, the compositions disclosed herein, such as hair compositions, make it possible to obtain a high sheen immediately after application thereof, e.g., immediately after the treatment of a keratinous substance.

However, in contrast to known compositions which comprise different metal particles from those incorporated in the compositions disclosed herein, i.e., non-encapsulated metal particles, the high sheen obtained with the compositions disclosed herein can be retained for a prolonged period of time.

By way of example, this high sheen can be maintained for a period of time which can reach, for example, one month or more for hair treated with the compositions disclosed herein, whereas a known composition, such as that disclosed in the document EP-A-1 064 918, including particles not in accordance with the invention, namely non-encapsulated particles, can lose all its sheen or reflectivity after a period of one month.

In addition to the retention of the sheen overtime, the cosmetic compositions disclosed herein can exhibit a markedly better stability over time than that of the known compositions, for example disclosed in the document EP-A-1 064 918, which comprise different metal particles from those included in the compositions disclosed herein, for example, non-encapsulated metal particles.

It would appear, but without wishing to be committed to any theory, that the specific metal particles employed in the compositions disclosed herein can limit the aggregation of the metal nanoparticles in polar media, such as water and/or ethanol, and thus make it possible to obtain colloidal dispersions of high stability without phase separation.

In addition, the protection contributed by the shell of the at least one organic material may have the effect of preventing surface oxidation of the at least one metal predominantly constituting the particles under the effect of external agents, whether these are agents present in the composition or agents with which the particles are liable to be in contact when the composition is applied, such as sebum, sweat, tears, atmospheric agents, and the like.

By preventing the oxidation of the at least one metal of the particles, the loss in reflectivity of the metal and the loss in sheen which are the consequence of this oxidation can be avoided.

In one embodiment, a hair cosmetic composition, such as a hair cosmetic composition for contributing sheen to the hair, is disclosed.

Further disclosed herein is a cosmetic process for the treatment of keratinous substances, such as the hair, for example, for contributing sheen to the keratinous substances, comprising applying to the keratinous substances or fibers, such as the hair, the composition as described above.

Even further disclosed herein is the use of the composition as described above for contributing sheen to keratinous substances, such as the hair.

Even further disclosed herein is the use of the specific particles as described herein in a cosmetic composition for contributing sheen to keratinous substances or fibers, such as the hair.

The disclosure will now be described in more detail as follows.

The cosmetic compositions disclosed herein comprise at least one agent exhibiting a cosmetic activity or having a cosmetic effect.

The term “agent exhibiting a cosmetic activity” or “cosmetic active principle”, as used herein, means any active compound having a cosmetic or dermatological activity or alternatively any compound capable of modifying the appearance, the feel and/or the physicochemical properties of keratinous substances, such as the hair.

The at least one agent exhibiting a cosmetic activity (the at least one cosmetic active principle) as disclosed herein may be chosen, for example, from:
saccharides, oligosaccharides and polysaccharides which may optionally be hydrolyzed and/or modified,
amino acids, oligopeptides, peptides, proteins, which may optionally be hydrolyzed and/or modified, poly(amino acid)s and enzymes,
branched and unbranched fatty acids and alcohols,
animal, vegetable and mineral waxes,
ceramides and pseudoceramides,
hydroxylated organic acids,
UV screening agents,
antioxidants and agents for combating free radicals,
chelating agents,
antidandruff agents,
seborrhoea-regulating agents,
soothing agents,
cationic surfactants,
cationic and amphoteric polymers,
organomodified and non-organomodified silicones,
mineral, vegetable and animal oils,
polyisobutenes and poly(α-olefin)s,
esters,
soluble and dispersed anionic polymers,
soluble and dispersed non-ionic polymers,
reducing agents,
coloring agents and coloring materials, such as hair dyes,
foaming agents,
film-forming agents,
particles (other than the particles having a core-shell structure as disclosed herein), and the mixtures thereof.

This agent exhibiting a cosmetic activity is present in a concentration ranging from 0.001% to 10% by weight relative to the total weight of the cosmetic composition, such as from 0.01% to 5% by weight relative to the total weight of the cosmetic composition.

The compounds of saccharide, oligosaccharide or polysaccharide which may optionally be hydrolyzed and/or modified and can be used herein are chosen from those which are described, for example, in “Encyclopedia of Chemical Technology, Kirk-Othmer, Third Edition, 1982, volume 3, pp. 896-900, and volume 15, pp. 439-458”, in “Polymers in Nature, by E. A. MacGregor and C. T. Greenwood, published by John Wiley & Sons, Chapter 6, pp. 240-328,1980” and in “Industrial Gums—Polysaccharides and their Derivatives, edited by Roy L. Whistler, Second Edition, published by Academic Press Inc.”

Mention may, for example, be made, as examples of saccharides, oligosaccharides or polysaccharides which may optionally be hydrolyzed and/or modified and can be used herein, of glucans, modified or unmodified starches (such as those resulting, for example, from cereals, such as wheat, maize and rice, from vegetables, such as yellow split peas, and from tubers, such as potatoes and manioc) which are different from starch betainate (starch as described above), amylose, amylopectin, glycogen, dextrans, β-glucans, celluloses and their derivatives (methylcelluloses, hydroxyalkyl-celluloses, ethylhydroxyethylcelluloses and carboxymethylcelluloses), fructosans, inulin, levan, mannans, xylans, lignins, arabans, galactans, galacturonans, chitin, glucoronoxylans, arabinoxylans, xyloglucans, galactomannans, glucomannans, pectic acids and pectins, alginic acid and alginates, arabinogalactans, carrageenans, agars, glycosaminoglucans, gums arabic, gums tragacanth, ghatti gums, karaya gums, locust bean gums, guar gums and xanthan gums.

Mention may be made, as amino acids, of, for example, cysteine, lysine, alanine, N-phenylalanine, arginine, glycine, leucine, and their mixtures. Mention may, for example, be made, as oligopeptides, peptides and proteins which may optionally be hydrolyzed and/or modified and can be used herein, of hydrolysates of wool or silk proteins, which may optionally be modified, and plant proteins, such as wheat proteins.

Mention may be made, among poly(amino acid)s which can be used, of polylysine.

Mention may be made, among enzymes which can be used, of laccases, peroxidases, lipases, proteases, glycosidases, dextranases, uricases and alkaline phosphatase.

Mention may, for example, be made, among branched and unbranched fatty acids suitable to be used herein, of C₈-C₃₀ carboxylic acids, such as palmitic acid, oleic acid, linoleic acid, myristic acid, stearic acid, lauric acid, and their mixtures. The fatty alcohols which can be used herein comprise, for example, C₈-C₃₀ alcohols, such as palmityl, oleyl, linoleyl, myristyl, stearyl and lauryl alcohols.

A wax as used herein is a lipophilic compound, solid at ambient temperature (approximately 25° C.), with a reversible solid/liquid change of state, having a melting point of greater than approximately 40° C. and which can range up to 200° C., and exhibiting, in the solid state, an anisotropic crystalline arrangement. The size of the crystals of the wax may be such that the crystals diffract and/or scatter light, conferring on the composition which comprises them a cloudy appearance which is more or less opaque. On bringing the wax to its melting point, it is possible to render it miscible with oils and to form a microscopically homogeneous mixture but, on bringing the temperature of the mixture back to ambient temperature, a recrystallization of the wax from the oils of the mixture is obtained which is detectable microscopically and macroscopically (opalescence).

Mention may be made, as waxes which can be used herein, of waxes of animal origin, such as beeswax, spermaceti, lanolin wax and lanolin derivatives; vegetable waxes, such as carnauba wax, candelilla wax, ouricury wax, Japan wax, cocoa butter and cork fibre and sugarcane waxes; and mineral waxes, for example, paraffin wax, petrolatum wax, lignite wax, microcrystalline waxes and ozokerites.

Mention may, for example, be made, among ceramides, of ceramides of the classes I, II, III and V according to the Downing classification, for example, N-oleyldehydrosphingosine.

The hydroxylated organic acids are chosen from those well known and used in the art. Mention may, for example, be made of citric acid, lactic acid, tartaric acid and malic acid.

The sunscreens active in the UV-A and/or UV-B regions which can be used herein are those well known to a person skilled in the art. Mention may, for example, be made of dibenzoylmethane derivatives, such as 4-methyldibenzoylmethane, 4-isopropyldibenzoylmethane, 4-tert-butyldibenzoylmethane, 2,4-dimethyldibenzoylmethane and 4-tert-butyl-4′-diisopropyldibenzoylmethane, p-aminobenzoic acid and its esters, such as 2-ethylhexyl p-dimethylaminobenzoate and N-propoxylated ethyl p-aminobenzoate, salicylates, such as triethanolamine salicylate, cinnamic acid esters, such as 2-ethylhexyl 4-methoxycinnamate and methyl diisopropylcinnamate, menthyl anthranilate, benzotriazole derivatives, triazine derivatives, β,β-diphenylacrylate derivatives, such as 2-ethylhexyl 2-cyano-3,3-diphenylacrylate and ethyl 2-cyano-3,3-diphenylacrylate, 2-phenylbenzimidazole-5-sulphonic acid and its salts, benzophenone derivatives, benzylidenecamphor derivatives, silicone-comprising screening agents, and the like.

Mention may be made, as antioxidants and agents for combating free radicals which can be used herein, of, for example, ascorbic acid, ascorbylated compounds, such as ascorbyl dipalmitate, t-butylhydroquinone, polyphenols, such as phloroglucinol, sodium sulphite, erythorbic acid and flavonoids.

The chelating agents can be chosen, for example, from EDTA (ethylenediaminetetraacetic acid) and its salts, such as disodium EDTA and dipotassium EDTA, phosphate-comprising compounds, such as sodium metaphosphate, sodium hexametaphosphate and tetrapotassium pyrophosphate, and phosphonic acids and their salts, such as the salts of ethylenediaminetetramethylenephosphonic acid.

The antidandruff agents are chosen, for example, from:
benzethonium chloride, benzalkonium chloride, chlorhexidine, chloramine-T, chloramine-B, 1,3-dibromo-5,5-dimethylhydantoin, 1,3-dichloro-5,5-di-methylhydantoin, 3-bromo-1-chloro-5,5-dimethylhydantoin and N-chlorosuccinimide; 1-hydroxy-2-pyridone derivatives, such as 1-hydroxy-4-methyl-2-pyridone, 1-hydroxy-6-methyl-2-pyridone and 1-hydroxy-4,6-dimethyl-2-pyridone;
trihalocarbamides;
triclosan;
azole-comprising compounds, such as climbazole, ketoconazole, clotrimazole, econazole, isoconazole and miconazole b;
antifungal polymers, such as amphotericin B and nystatin;
selenium sulphides; and
sulphur in its various forms, cadmium sulphide, allantoin, coal and wood tars and their derivatives, such as oil of cade, undecylenic acid, fumaric acid, and allylamines, such as terbinafine.

They may also be used in the form of their addition salts with physiologically acceptable acids, such as in the form of salts of sulphuric, nitric, thiocyanic, hydrochloric, hydrobromic, hydriodic, phosphoric, acetic, benzoic, glycolic, aceturic, succinic, nicotinic, tartaric, maleic, palmitic, methanesulphonic, propanoic, 2-oxopropanoic, propanedioic, 2-hydroxy-1,4-butanedioic, 3-phenyl-2-propenoic, α-hydroxybenzeneacetic, ethanesulphonic, 2-hydroxyethanesulphonic, 4-methylbenzenesulphonic, 4-amino-2-hydroxybenzoic, 2-phenoxybenzoic, 2-acetyloxybenzoic, picric, lactic, citric, malic and oxalic acids and of amino acids.

The antidandruff agents mentioned above can also, if appropriate, be used in the form of their addition salts with physiologically acceptable organic or inorganic bases. Examples of organic bases include alkanolamines with low molecular weights, such as ethanolamine, diethanolamine, N-ethylethanolamine, triethanolamine, diethylaminoethanol and 2-amino-2-methylpropanedione; nonvolatile bases, such as ethylenediamine, hexamethylenediamine, cyclohexylamine, benzylamine and N-methylpiperazine; quaternary ammonium hydroxides, for example trimethylbenzylammonium hydroxide; and guanidine and its derivatives, such as its alkylated derivatives. Examples of inorganic bases include the salts of alkali metals, such as sodium and potassium; ammonium salts; the salts of alkaline earth metals, such as magnesium and calcium; and the salts of cationic di-, tri- and tetravalent metals, such as zinc, aluminium and zirconium. For example, alkanolamines, ethylenediamine and inorganic bases, such as the salts of alkali metals, can be used.

The seborrhoea-regulating agents are, for example, succinylchitosan and poly-β-alanine.

The soothing agents are, for example, azulene and glycyrrhetinic acid.

The cationic surfactants are those well known per se, such as salts of primary, secondary and tertiary fatty amines which are optionally polyoxyalkylenated; quaternary ammonium salts, such as tetraalkylammonium, alkyl-amidoalkyltrialkylammonium, trialkylbenzylammonium, trialkylhydroxyalkylammonium and alkylpyridinium chlorides and bromides; and imidazoline derivatives.

The term “cationic polymer” as used herein means any polymer comprising cationic groups and/or groups which can be ionized to cationic groups.

The cationic polymers which can be used herein can be chosen from all those already known per se as improving the cosmetic properties of hair treated with detergent compositions, for example, those disclosed in Patent Application EP-A-0 337 354 and in French Patent Applications FR-A-2 270 846, 2 383 660, 2 598 611, 2 470 596 and 2 519 863.

The cationic polymers are chosen, for example, from those which comprise units comprising at least one amine group chosen from primary, secondary, tertiary and quaternary amine groups which can either form part of the main polymer chain or be carried by a side substituent directly connected to the main chain.

The cationic polymers used herein may have a number-average molecular mass ranging from 500 to 5×10⁶ approximately such as from 10³ to 3×10⁶ approximately.

Mention may, for example, be made, among cationic polymers, of the polymers of the polyamine, polyaminoamide and poly(quaternary ammonium) type. These are known products.

The polymers of the polyamine, polyaminoamide and poly(quaternary ammonium) types which can be used in the composition disclosed herein include those disclosed in French Patents Nos. 2 505 348 and 2 542 997. Mention may be made, among these polymers, of:
(1) homopolymers or copolymers derived from esters or amides of acrylic and methacrylic acids;
(2) cellulose ether derivatives comprising quaternary ammonium groups disclosed in French Patent No. 1 492 597;
(3) cationic cellulose derivatives, such as the copolymers of cellulose and the cellulose derivatives grafted with a water-soluble quaternary ammonium monomer and disclosed, for example, in U.S. Pat. No. 4,131,576, such as hydroxyalkylcelluloses, for example hydroxymethyl-, hydroxyethyl- and hydroxypropyl-celluloses, grafted, for example, with a methacryloylethyltrimethylammonium, methacrylamidopropyltrimethylammonium or dimethyldiallylammonium salt;
(4) the cationic polysaccharides disclosed, for example, in U.S. Pat. Nos. 3,589,578 and 4,031,307, such as guar gums comprising trialkylammonium cationic groups;
(5) polymers comprising piperazinyl units and at least one group chosen from divalent, straight- and branched-chain alkylene and hydroxyalkylene groups, optionally interrupted by at least one entity chosen from oxygen, sulphur and nitrogen atoms and aromatic and heterocyclic rings, as well as the oxidation and/or quaternization products of these polymers. Such polymers are disclosed, for example, in French Patent Nos. 2 162 025 and 2 280 361;
(6) water-soluble polyaminoamides, such as those disclosed, for example, in French Patent Nos. 2 252 840 and 2 368 508;
(7) polyaminoamide derivatives, for example the adipic acid/dialkylaminohydroxyalkyldialkylenetriamine polymers in which the alkyl group comprises from 1 to 4 carbon atoms such as methyl, ethyl and propyl groups and the alkylene group comprises from 1 to 4 carbon atoms such as an ethylene group. Such polymers are disclosed, for example, in French Patent No. 1 583 363;
(8) polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms. The molar ratio of polyalkylenepolyamine to dicarboxylic acid ranges from 0.8:1 to 1.4:1; wherein the polyaminoamide resulting therefrom is reacted with epichlorohydrin in a molar ratio of epichlorohydrin with respect to the secondary amine group of the polyaminoamide ranging from 0.5:1 to 1.8:1. Such polymers are disclosed, for example, in U.S. Pat. Nos. 3,227,615 and 2,961,347;
(9) cyclopolymers of alkyldiallylamine or of dialkyldiallylammonium, such as the homopolymer of dimethyldiallylammonium chloride and the copolymers of diallyldimethylammonium chloride and of acrylamide;
(10) the quaternary diammonium polymers exhibiting a number-average molecular mass ranging from 1000 to 100 000, such as those disclosed, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020;
(11) polymers of poly(quaternary ammonium)s, such as those disclosed, for example, in Patent Application EP-A-122 324;
(12) quaternary polymers of vinylpyrrolidone and of vinylimidazole, such as the products sold under the names Luviquat® FC 905, FC 550 and FC 370 by BASF;
(13) polyamines, such as Polyquart® H sold by Henkel, referenced under the name of “Polyethylene Glycol (15) Tallow Polyamine” in the CTFA dictionary;
(14) crosslinked polymers of methacryloyloxy(C₁-C₄)alkyltri(C₁-C₄)alkylammonium salts, such as those sold under the names of Salcare® SC 92, Salcare® SC 95 and Salcare® SC 96 by Allied Colloids; and
   the mixtures thereof.

Other cationic polymers which can be used herein include cationic proteins, cationic protein hydrolysates, polyalkyleneimines, such as polyethyleneimines, polymers comprising vinylpyridine or vinylpyridinium units, condensates of polyamines and of epichlorohydrin, quaternary polyureylenes and chitin derivatives.

The amphoteric polymers which can be used herein can be chosen from polymers comprising B and C units distributed randomly in the polymer chain, wherein the B unit is a unit deriving from a monomer comprising at least one basic nitrogen atom and the C unit is a unit deriving from an acidic monomer comprising at least one group chosen from carboxyl and sulpho groups or else B and C units can be chosen from groups deriving from zwitterionic carboxybetaine and sulphobetaine monomers; B and C units can also be chosen from cationic polymer chains comprising at least one amine group chosen from primary, secondary, tertiary and quaternary amine groups, in which at least one of the amine groups carries a carboxyl or sulpho group connected via a hydrocarbonaceous group, or else B and C units form part of a chain of a polymer comprising a dicarboxyethylene unit, one of the carboxyl groups of which has been reacted with a polyamine comprising at least one amine group chosen from primary and secondary amine groups.

The amphoteric polymers corresponding to the definition given above may, for example, be chosen from the following polymers:
(1) polymers resulting from the copolymerization of a monomer derived from a vinyl compound carrying at least one carboxyl group, such as acrylic acid, methacrylic acid, maleic acid and α-chloroacrylic acid, and of a basic monomer derived from a substituted vinyl compound comprising at least one basic atom, such as dialkylaminoalkyl methacrylates and acrylates and dialkylaminoalkylmethacrylamides and -acrylamides. Such compounds are disclosed for example, in U.S. Pat. No. 3,836,537. Mention may also be made of the sodium acrylate/acrylamidopropyltrimethylammonium chloride copolymer sold under the name Polyquart® KE 3033 by Henkel.

The vinyl compound can also be a dialkyldiallylammonium salt, such as diethyldiallylammonium chloride.

Copolymers of acrylic acid and of the basic monomer are provided, for example, under the names Merquat® 280, Merquat® 295 and Merquat® Plus 3330 by Calgon.
(2) polymers comprising units deriving:
a) from at least one monomer chosen from acrylamides and methacrylamides substituted on the nitrogen by an alkyl group,
b) from at least one acidic comonomer comprising at least one reactive carboxyl group, and
c) from at least one basic comonomer, such as esters comprising primary, secondary, tertiary and quaternary amine substituents of acrylic and methacrylic acids and the quaternization product of dimethylaminoethyl methacrylate with dimethyl or diethyl sulphate.

For example, the N-substituted acrylamides and methacrylamides disclosed herein include the groups in which the alkyl groups comprise from 2 to 12 carbon atoms such as N-ethylacrylamide, N-tert-butylacrylamide, N-tert-octylacrylamide, N-octylacrylamide, N-decylacrylamide and N-dodecylacrylamide, and the corresponding methacrylamides.

The acidic comonomers are, for example, chosen from acrylic, methacrylic, crotonic, itaconic, maleic and fumaric acids and alkyl monoesters comprising from 1 to 4 carbon atoms of maleic or fumaric acids or anhydrides. The basic comonomers are, for example, chosen from aminoethyl, butylaminoethyl, N,N-dimethylaminoethyl and N-tert-butylaminoethyl methacrylates. The copolymers for which the CTFA name (4th Ed., 1991) is octylacrylamide/acrylates/butylaminoethyl methacrylate copolymer, such as the products sold under the name Amphomer® or Lovocryl® 47 by National Starch can, for example, be used.
(3) partially or completely alkylated and crosslinked polyaminoamides deriving from polyaminoamides of formula:
   CO—R₁₀—C-Z (II)
   wherein R₁₀ is a divalent group derived from a saturated dicarboxylic acid, from an aliphatic mono- or dicarboxylic acid comprising an ethylenic double bond, from an ester of a lower alkanol having from 1 to 6 carbon atoms of these acids, or from a group deriving from the addition of any one of the acids with a bisprimary or bis(secondary derived) amine, and Z is a group chosen from groups of bisprimary, mono- and bissecondary polyalkylenepolyamines and, for example, Z represents:
a) in proportions ranging from 60 mol % to 100 mol %, the group
   —NHCH₂)ₓ—NHₚ (III)
   wherein x=2 and p=2 or 3, or else x=3 and p=2 wherein this group is derived from diethylenetriamine, triethylenetetraamine or dipropylenetriamine;
b) in proportions ranging from 0 to 40 mol %, the above group (III), wherein x=2 and p=1 and which derives from ethylenediamine, or the group deriving from piperazine: 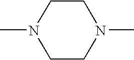
c) in proportions ranging from 0 to 20 mol %, the group —NH—(CH₂)₆—NH— deriving from hexamethylenediamine, wherein these polyaminoamides are crosslinked by addition of a bifunctional crosslinking agent chosen from epihalohydrins, diepoxides, dianhydrides and bisunsaturated derivatives, by means of from 0.025 to 0.35 mol of crosslinking agent per amine group of the polyaminoamide, and alkylated by reaction with acrylic acid, chloracetic acid or an alkanesultone or their salts.

The saturated carboxylic acids are, for example, chosen from acids having from 6 to 10 carbon atoms, such as adipic, 2,2,4-trimethyladipic and 2,4,4-trimethyladipic, and terephthalic acids, and the acids comprising an ethylenic double bond, such as acrylic, methacrylic and itaconic acids. The alkanesultones used in the alkylation are chosen, for example, from propane- and butanesultone and the salts of the alkylating agents are, for example, chosen from the sodium and potassium salts.
(4) polymers comprising zwitterionic units of formula (IV): 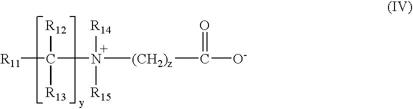
   wherein R₁₁ is a polymerizable unsaturated group, such as an acrylate, methacrylate, acrylamide or methacrylamide group, y and z are each an integer from 1 to 3, R₁₂ and R₁₃, which may be identical or different, are each chosen from a hydrogen atom and methyl, ethyl and propyl groups, and R₁₄ and R₁₅, which may be identical or different, are each chosen from a hydrogen atom and alkyl groups such that the sum of the carbon atoms in R₁₄ and R₁₅ does not exceed 10.

The polymers comprising such units can also comprise units derived from non-zwitterionic monomers, such as dimethyl- and diethylaminoethyl acrylate and methacrylate, alkyl acrylates and methacrylates, acrylamides and methacrylamides, and vinyl acetate.

Mention may be made, by way of example, of the copolymer of methyl methacrylate and of dimethylcarboxymethylammonioethyl methacrylate, such as the product sold under the name Diaformer® Z301 by Sandoz.
(5) polymers derived from chitosan comprising monomer units corresponding to the following formulae: 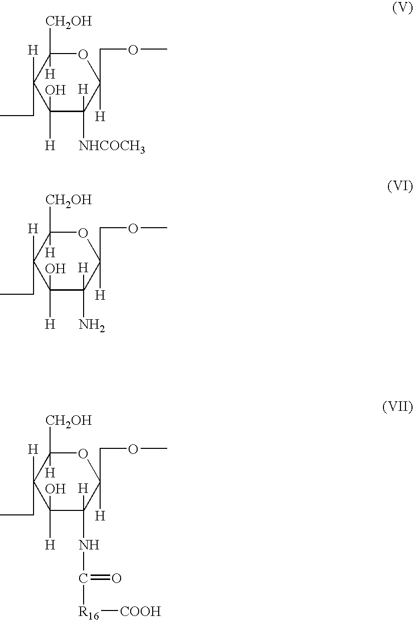
   wherein the unit (V) is present in a proportion ranging from 0 to 30%, the unit (VI) in a proportion ranging from 5 to 50% and the unit (VII) in a proportion ranging from 30 to 90%, wherein in the unit (VII), R₁₆ is a group of formula (VIII): 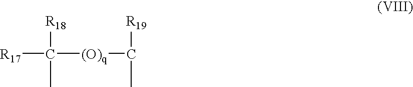
   wherein, if q=0, R₁₇, R₁₈ and R₁₉, which are identical or different, each are chosen from a hydrogen atom, methyl, hydroxyl, acetoxy and amino residues, monoalkylamino residues and dialkylamino residues, optionally interrupted by at least one nitrogen atom and/or optionally substituted by at least one group chosen from amino, hydroxyl, carboxyl, alkylthio and sulpho groups, and alkylthio residues in which the alkyl group carries an amino residue, wherein at least one of the R₁₇, R₁₈ and R₁₉ groups is, in this case, a hydrogen atom;
or, if q=1, R₁₇, R₁₈ and R₁₉ are each a hydrogen atom,
and the salts formed by these compounds with bases or acids.
(6) polymers derived from the N-carboxyalkylation of chitosan, such as the N-(carboxymethyl)chitosan and the N-(carboxybutyl)chitosan sold under the name Evalsan® by Jan Dekker.
(7) polymers corresponding to the formula (IX) disclosed, for example, in French Patent No. 1 400 366: 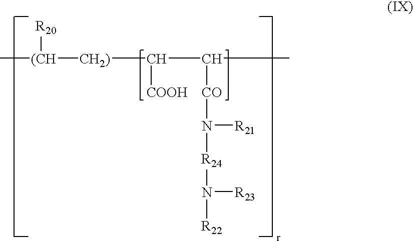
   wherein R₂₀ is chosen from a hydrogen atom and CH₃O, CH₃CH₂O and phenyl groups, R₂₁ is chosen from hydrogen and lower alkyl groups, such as methyl and ethyl, R₂₂ is chosen from hydrogen and lower alkyl groups, such as methyl and ethyl, R₂₃ is chosen from lower alkyl groups, such as methyl and ethyl, and a group corresponding to the formula: —R₂₄—N(R₂₂)₂, wherein R₂₄ is chosen from —CH₂—CH₂—, —CH₂—CH₂—CH₂— and —CH₂—CH(CH₃)— groups and R₂₂ has the same meaning mentioned above; and R₂₄ has the same meaning mentioned above,
as well as the higher homologues of these groups comprising up to 6 carbon atoms.
(8) amphoteric polymers of the -D-X₁-D-X₁— type chosen from:
a) polymers obtained by reaction of chloroacetic acid or sodium chloroacetate with compounds comprising at least one unit of formula:
   -D-X₁-D-X₁-D- (X)
   wherein D is a group 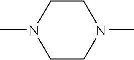
   and X₁ is chosen from the symbols E and E′, wherein E and E′, which are identical or different, are each chosen from divalent alkylene groups comprising at least one chain chosen from straight- and branched-chains comprising up to 7 carbon atoms in the main chain, wherein the divalent alkylene groups are optionally substituted by at least one hydroxyl group. E or E′ can additionally comprise at least one entity chosen from oxygen, nitrogen and sulphur atoms and 1 to 3 aromatic and heterocyclic rings. The oxygen, nitrogen and sulphur atoms can be present in the form of at least one group chosen from ether, thioether, sulphoxide, sulphone, sulphonium, alkylamine and alkenylamine groups, and hydroxyl, benzylamine, amine oxide, quaternary ammonium, amide, imide, alcohol, ester and urethane groups.
b) polymers of formula:
   -D-X₁-D-X₁— (XI)
   wherein D is a group 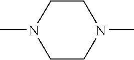
   and X₁ is chosen from the symbols E and E′ and wherein at least one X₁ is chosen from E′, E having the meaning indicated above and E′ being chosen from divalent alkylene groups comprising at least one chain chosen from straight- and branched chains comprising up to 7 carbon atoms in the main chain, wherein the divalent alkylene groups are optionally substituted by at least one hydroxyl group. E′ can also comprise at least one nitrogen atom substituted by an alkyl chain which is optionally interrupted by an oxygen atom, wherein the alkyl chain comprises at least one functional group chosen from carboxyl functional groups and hydroxyl functional groups and wherein the alkyl chain is betainized by reaction with a reactant chosen from chloroacetic acid and sodium chloroacetate.
(9) (C₁-C₅)alkyl vinyl ether/maleic anhydride copolymers partially modified by semiamidation with an N,N-dialkylaminoalkylamine, such as N,N-dimethylamino-propylamine, or by semiesterification with an N,N-dialkanolamine. These copolymers can also comprise other vinyl comonomers, such as vinylcaprolactam.

The silicones which can be used herein can be soluble or insoluble in water and they can, for example, be polyorganosiloxanes which are insoluble in water; they can be provided in the form of oils, of waxes, of resins or of gums.

Organopolysiloxanes are defined in more detail in the work by Walter Noll, “Chemistry and Technology of Silicones” (1968), Academic Press. They can be volatile or non-volatile.

When they are volatile, the silicones can be chosen, for example, from those having a boiling point ranging from 60° C. to 260° C. and further, for example, be chosen from:
(i) cyclic silicones comprising from 3 to 7 silicon atoms such as from 4 to 5 silicon atoms. An example is octamethylcyclotetrasiloxane, sold, for example, under the name of “Volatile Silicone 7207” by Union Carbide or “Silbione 70045 V 2” by Rhodia, or decamethylcyclopentasiloxane, sold under the name of “Volatile Silicone 7158” by Union Carbide or “Silbione 70045 V 5” by Rhodia.

Mention may also be made of cyclocopolymers of the dimethylsiloxane/methylalkylsiloxane type, such as “Silicone Volatile FZ 3109”, sold by Union Carbide, with the chemical structure:
with D: 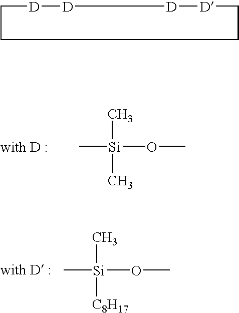
(ii) linear volatile silicones comprising from 2 to 9 silicon atoms and having a viscosity of less than or equal to 5×10⁻⁶ m²/s at 25° C. An example is decamethyltetrasiloxane, sold, for example, under the name “SH 200” by Toray Silicone. Silicones coming within this class are also described in the article published in Cosmetics and Toiletries, Vol. 91, Jan. 76, p. 27-32, Todd & Byers, “Volatile Silicone Fluids for Cosmetics”.

Mention may, for example, be made, among nonvolatile silicones, of polyalkylsiloxanes, polyarylsiloxanes, polyalkylarylsiloxanes, silicone gums and resins, and polyorganosiloxanes modified by organofunctional groups.

The organomodified silicones which can be used herein include silicones as defined above and comprising, in their structure, at least one organofunctional group attached via a hydrocarbonaceous group.

Mention may be made, among the organomodified silicones, of polyorganosiloxanes comprising:
at least one group chosen from polyethyleneoxy and polypropyleneoxy groups optionally comprising at least one alkyl group chosen from C₆-C₂₄ alkyl groups, such as the products known as dimethicone copolyol sold by Dow Corning under the name DC 1248, the Silwet® L 722, L 7500, L 77 and L 711 oils from Union Carbide and the (C₁₂)alkyl methicone copolyol sold by Dow Corning under the name Q2 5200;
substituted or unsubstituted amino groups, such as the products sold under the names GP 4 Silicone Fluid and GP 7100 by Genesee and the products sold under the names Q2 8220 and Dow Corning 929 or 939 by Dow Corning. The substituted amino groups are, for example, chosen from C₁-C₄ aminoalkyl groups;
thiol groups, such as the products sold under the names “GP 72 A” and “GP 71” from Genesee;
alkoxylated groups, such as the product sold under the names “Silicone Copolymer F-755” by SWS Silicones and Abil Wax® 2428, 2434 and 2440 by Goldschmidt;
hydroxylated groups, such as the polyorganosiloxanes comprising a hydroxyalkyl functional group disclosed in French Patent Application FR-A-85 16334;
acyloxyalkyl groups, such as the polyorganosiloxanes disclosed in U.S. Pat. No. 4,957,732;
anionic groups of the carboxylic acid type, such as in the products disclosed in Patent EP 186 507 from Chisso Corporation, or of the alkylcarboxylic type, such as those present in the product X-22-3701 E from Shin-Etsu; 2-hydroxyalkylsulphonate or 2-hydroxyalkyl thiosulphate, such as the products sold by Goldschmidt under the names “Abil® S201” and: “Abil® S255”;
hydroxyacylamino groups, such as the polyorganosiloxanes disclosed in application EP 342 834. Mention may be made, for example, of the product Q2-8413 from Dow Corning.

Mention may, for example, be made, as oils of vegetable origin, of sweet almond oil, avocado oil, castor oil, olive oil, jojoba oil, sunflower oil, wheat germ oil, sesame oil, groundnut oil, grape seed oil, soybean oil, rapeseed oil, safflower oil, coconut oil, maize oil, hazelnut oil, karite butter, palm oil, apricot kernel oil and calophyllum oil; as oils of animal origin, of perhydrosqualene; as oils of mineral origin, of liquid paraffin and liquid petrolatum.

The polyisobutenes and poly(α-olefin)s are chosen from those well known in the art.

Mention may, for example, be made, as examples of esters, of esters of fatty acids, such as isopropyl myristate, isopropyl palmitate, 2-ethylhexyl palmitate, purcellin oil (stearyl octanoate), isononyl isononanoate, isostearyl isononanoate, isopropyl lanolate, and their mixtures.

The anionic polymers useful herein are polymers comprising groups derived from acids chosen from carboxylic, sulphonic and phosphoric acids and exhibiting a weight-average molecular mass ranging from 500 to 5 000 000.

The carboxyl groups are contributed by unsaturated carboxylic monoacid or diacid monomers, such as those corresponding to the formula: 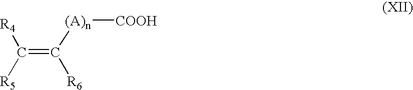
wherein n is an integer from 0 to 10, A is a methylene group, optionally connected to the carbon atom of the unsaturated group or to the neighboring methylene group when n is greater than 1 via a heteroatom, such as oxygen and sulphur, R₄ is chosen from a hydrogen atom and phenyl and benzyl groups, R₅ is chosen from a hydrogen atom and lower alkyl and carboxyl groups, and R₆ is chosen from a hydrogen atom, lower alkyl groups, —CH₂—COOH, phenyl and benzyl groups.

In the above formula (XII), the lower alkyl group may comprise, for example, from 1 to 4 carbon atoms such as methyl and ethyl groups.

In one embodiment, the anionic polymers comprising carboxyl groups disclosed herein include:

A) Homo- or copolymers of acrylic or methacrylic acid or their salts such as the products sold under the names Versicol® E or K by Allied Colloid and Ultrahold® by BASF, the copolymers of acrylic acid and of acrylamide sold in the form of their sodium salt under the names Reten® 421, 423 and 425 by Hercules and the sodium salts of polyhydroxy-carboxylic acids.

B) Copolymers of acrylic acid or methacrylic acid with a monoethylenic monomer, such as ethylene, styrene, vinyl esters and esters of acrylic and methacrylic acid, optionally grafted onto a polyalkylene glycol, such as polyethylene glycol, and optionally crosslinked. Such polymers are disclosed, for example, in French Patent No. 1 222 944 and German Application No. 2 330 956, the copolymers of this type comprising, in their chain, an optionally N-alkylated and/or -hydroxyalkylated acrylamide unit, such as disclosed in, for example, Luxembourgian Patent Application Nos. 75370 and 75371 and provided under the name Quadramer® by American Cyanamid. Mention may also be made of copolymers of acrylic acid and of C₁-C₄ alkyl methacrylate and the copolymer of methacrylic acid and of ethyl acrylate sold under the name Luvimer® MAEX by BASF.

C) Copolymers derived from crotonic acid, such as those comprising, in their chain, vinyl acetate or propionate units and optionally other monomers, such as allyl and methallyl esters, vinyl ether and vinyl esters of a linear or branched saturated carboxylic acid comprising a long hydrocarbonaceous chain, such as those comprising at least 5 carbon atoms, it optionally being possible for these polymers to be grafted and crosslinked, or alternatively a vinyl, allyl or methallyl ester of an α- or β-cyclic carboxylic acid. Such polymers are disclosed, inter alia, in French Patent Nos. 1 222 944, 1 580 545, 2 265 782, 2 265 781, 1 564 110 and 2 439 798. Commercial products coming within this class include the Resins 28-29-30, 26-13-14 and 28-13-10 sold by National Starch.

D) Polymers derived from maleic, fumaric or itaconic acids or anhydrides with vinyl esters, vinyl ethers, vinyl halides, phenylvinyl derivatives, or acrylic acid and its esters; these polymers can be esterified. Such polymers are disclosed, for example, in U.S. Pat. Nos. 2,047,398, 2,723,248 and 2,102,113 and Patent GB 839 805 such as those sold under the names Gantrez® AN and ES by ISP.

Polymers also coming within this class are copolymers of maleic, citraconic or itaconic anhydrides and of an allyl or methallyl ester, optionally comprising an acrylamide or methacrylamide group, an α-olefin, acrylic or methacrylic esters, acrylic or methacrylic acids, or vinylpyrrolidone in their chain; the anhydride functional groups are monoesterified or monoamidated. These polymers are, for example, disclosed in French Patent Nos. 2 350 384 and 2 357 241.

E) Polyacrylamides comprising carboxylate groups.

The polymers comprising sulpho groups are polymers comprising vinylsulphonic, styrenesulphonic, naphthalenesulphonic or acrylamidoalkylsulphonic units.

These polymers can, for example, be chosen from:
salts of polyvinylsulphonic acid having a molecular mass ranging from approximately 1000 to approximately 100 000, as well as copolymers with an unsaturated comonomer, such as acrylic and methacrylic acids and their esters, as well as acrylamide and its derivatives, vinyl ethers and vinylpyrrolidone;
salts of polystyrenesulphonic acid, the sodium salts having a molecular mass of approximately 500 000 and of approximately 100 000 sold respectively under the names Flexan® 500 and Flexan® 130 by National Starch. These compounds are disclosed in French Patent No. 2 198 719;
salts of polyacrylamidosulphonic acids, such as those mentioned in U.S. Pat. No. 4,128,631 such as the polyacrylamidoethylpropanesulphonic acid sold under the name Cosmedia Polymer® HSP 1180 by Henkel.

As disclosed herein, the anionic polymers are, for example, chosen from acrylic acid copolymers, such as the acrylic acid/ethyl acrylate/N-tert-butylacrylamide terpolymer sold under the name Ultrahold Strong® by BASF, copolymers derived from crotonic acid, such as the vinyl acetate/vinyl tert-butylbenzoate/crotonic acid terpolymers and the crotonic acid/vinyl acetate/vinyl neododecanoate terpolymers sold under the name Resin 28-29-30 by National Starch, polymers derived from maleic, fumaric and itaconic acids and anhydrides with vinyl esters, vinyl ethers, vinyl halides, phenylvinyl derivatives, and acrylic acid and its esters, such as the monoesterified methyl vinyl ether/maleic anhydride copolymer sold under the name Gantrez® ES 425 by ISP, copolymers of methacrylic acid and of methyl methacrylate sold under the name Eudragit® L by Rohm Pharma, the copolymer of methacrylic acid and of ethyl acrylate sold under the name Luvimer® MAEX by BASF, the vinyl acetate/crotonic acid copolymer sold under the name Luviset® CA 66 by BASF and the vinyl acetate/crotonic acid/polyethylene glycol terpolymer sold under the name Aristoflex® A by BASF.

As disclosed herein, the anionic polymers can also be used in the latex or pseudolatex form, i.e., in the form of an aqueous dispersion of insoluble polymer particles.

Mention may, for example, be made, as non-ionic polymers which can be used herein, of:
vinylpyrrolidone homopolymers;
copolymers of vinylpyrrolidone and of vinyl acetate;
polyalkyloxazolines, such as the polyethyloxazolines provided by Dow Chemical under the names PEOX® 50 000, PEOX® 200 000 and PEOX® 500 000; vinyl acetate homopolymers, such as the product provided under the name Appretan® EM by Hoechst and the product provided under the name RhodopasS A 012 by Rhône-Poulenc; copolymers of vinyl acetate and of acrylic ester, such as the product provided under the name Rhodopas® AD 310 from Rhône-Poulenc; copolymers of vinyl acetate and of ethylene, such as the product provided under the name Appretan® TV by Hoechst;
copolymers of vinyl acetate and of maleic ester, for example of dibutyl maleate, such as the product provided under the name Appretan® MB Extra by Hoechst;
copolymers of polyethylene and of maleic anhydride;
alkyl acrylate homopolymers and alkyl methacrylate homopolymers, such as the product provided under the name Micropearl® RQ 750 by Matsumoto and the product provided under the name Luhydran® A 848 S by BASF;
acrylic ester copolymers, such as copolymers of alkyl acrylates and of alkyl methacrylates, such as the products provided by Rohm & Haas under the names Primal® AC-261 K and Eudragit® NE 30 D, by BASF under the names Acronal® 601, Luhydran® LR 8833 or 8845, and by Hoechst under the names Appretan® N 9213 and N9212;
copolymers of acrylonitrile and of a non-ionic monomer chosen, for example, from butadiene and alkyl (meth)acrylates; mention may be made of the products provided under the names Nipol® LX 531 8 by Nippon Zeon and those provided under the name CJ 0601 8 by Rohm & Haas;
polyurethanes, such as the products provided under the names Acrysol® RM 1020 and Acrysol® RM 2020 by Rohm & Haas and the products Uraflex® XP 401 UZ and Uraflex® XP 402 UZ provided by DSM Resins;
copolymers of alkyl acetate and of urethane, such as the product 8538-33 provided by National Starch;
polyamides, such as the product Estapor® LO 11 provided by Rhône-Poulenc; and
chemically modified or unmodified non-ionic guar gums.

The unmodified non-ionic guar gums are, for example, the products sold under the name Vidogum® GH 175 by Unipectine and under the name Jaguar® C by Meyhall.

The modified non-ionic guar gums which can be used may be, for example, modified with C₁-C₆ hydroxyalkyl groups. Mention may be made, for example, of the hydroxymethyl, hydroxyethyl, hydroxypropyl and hydroxybutyl groups.

These guar gums are well known in the state of the art and can, for example, be prepared by reacting corresponding alkene oxides, such as propylene oxides, with guar gum, so as to obtain a guar gum modified with hydroxypropyl groups.

Such non-ionic guar gums, optionally modified with hydroxyalkyl groups, are, for example, sold under the trade names Jaguar® HP8, Jaguar® HP60, Jaguar® HP120, Jaguar® DC 293 and Jaguar® HP 105 by Meyhall and under the name Galactasol® 4H₄FD2 by Aqualon.

The alkyl groups of the non-ionic polymers may comprise, for example, from 1 to 6 carbon atoms.

The reducing agents can be chosen from thioacids and their salts (thioglycolic acid or thiosulphate, cysteine or cysteamine), alkali metal and alkaline earth metal sulphites, reducing sugars, such as glucose, vitamin C and its derivatives, sulphovinic (ethyl sulphuric) acid derivatives, and phosphines.

The coloring agents can be chosen from linear and aromatic (heterocyclic and nonheterocylic) conjugated structures. Mention may be made, for example, of nitrobenzene dyes, aromatic dyes, aminobenzene dyes, azo dyes, anthraquinone dyes, aromatic diamines, aminophenols, phenols and naphthols, porphyrins, tetra-phenylporphyrins, metalloporphyrins, phthalocyanines, carotenoids, flavonoids and fluorescent molecules (fluorescein, rhodamine, coumarin, and the like).

The film-forming agents can be chosen from film-forming polymers, for example, those disclosed in documents FR-2 739 022, FR-2 757 048 and FR-2 767 699.

The foaming agents can be chosen from surfactants with a foaming nature and cationic polymers and anionic polymers with foaming properties; or alternatively the foaming agent can be a specific agent, such as that disclosed in document FR-2 751 221.

The particles, as cosmetic active principles, are other than the particles with a core-shell structure as disclosed herein and can be chosen from organic, inorganic and composite particles.

The cosmetic compositions as disclosed herein are essentially characterized by the particles which they comprise. These particles are, as disclosed herein, particles which can be defined as being metal nanoparticles encapsulated by an organic material.

As used herein, the term “nanoparticles” means particles with a size of less than or equal to 500 nm, such as ranging from 1 nm to 500 nm, further such as ranging from 1 nm to 100 nm, even further such as ranging from 1 nm to 50 nm.

The term “particle size” means the maximum dimension which it is possible to measure between two points of the particle. Such sizes can be measured directly by microscopic techniques, such as scanning electron microscopy and atomic force microscopy, or by indirect techniques, such as dynamic light scattering.

The particles incorporated in the compositions as disclosed herein can have various shapes. They can, for example, assume the shape of spheres, of flakes, of fibers, of tubes or of polyhedra. They can also have an entirely random shape. In one embodiment, the particles are spherical.

The particles incorporated in the compositions as disclosed herein have a core predominantly comprising at least one metal.

The term “metal” as used herein means a simple body composed solely of atoms of a metal element capable of generating cations.

The term “predominantly” as used herein means that the core of the particle comprises at least 50% by weight of the at least one metal.

For example, the core comprises at least 80% by weight, such as at least 90% by weight and further such as 100% by weight of the at least one metal.

The term “metal” as used herein means aluminium and all the elements with an atomic number ranging from 21 to 82 and composing Groups 3 to 13 of the Periodic Table of the Elements according to the new IUPAC notation: reference may be made, on this subject, to the CRC Handbook of Chemistry and Physics, 80th Print Edition.

The term “metal” as used herein also includes all the alloys of these elements, and the mixtures of these metals and alloys.

The core can thus also comprise, in the abovementioned percentages, a mixture of two or more of these metals and/or alloys thereof.

The core can also be a composite core comprising several regions, wherein adjacent regions comprise different metals, alloys and/or mixtures thereof.

In one embodiment, composite cores are multilayer cores comprising an inner core forming a substrate comprising at least one of metals, alloys and mixtures thereof, at least partially covered by a first layer comprising at least one of metals, alloys and mixtures thereof, which is different from that constituting the inner core, and optionally by at least one other layer, wherein each layer at least partially covers the preceding layer and each layer comprises at least one of metals, alloys and mixtures thereof, which is different from the following layer (if the latter exists) and from the preceding layer.

Apart from the at least one metal, the core can additionally comprise at least one stabilizer of any kind, and can also comprise unavoidable impurities.

In addition, the core can also comprise, for example, at least one metal compound other than the at least one metal, such as metal oxides.

Thus, in the case of aluminium, the core can comprise alumina Al₂O₃ in a proportion, for example, of 10% by weight per 90% of Al metal.

The at least one metal can, for example, be chosen from transition metals, rare earth metals and their alloys and mixtures thereof.

In one embodiment, the metal is chosen from aluminium, copper, silver, gold, indium, iron, platinum, nickel, molybdenum, titanium, tungsten, antimony, palladium, zinc, tin and their alloys and mixtures thereof.

In the preceding list, the metals referred to as “noble” and copper can, for example, be used. The term “noble metals” means gold, silver, palladium, platinum and their alloys and mixtures thereof.

In one embodiment, silver is used.

The solid shell can be in direct contact with the at least one metal which predominantly constitutes the core; in other words, no underlayer is inserted between the shell and the metal; or the core predominantly comprising at least one metal can, before it is encapsulated or before the formation of the shell, be modified at the surface by a treatment which modifies the properties of the core. This treatment can comprise stabilizing the surface of the core (i.e., the surface of the metal) by an adsorbed or covalently bonded monolayer.

As disclosed herein, the shell surrounding the core comprises at least one organic material.

The term “organic material” as used herein means any material comprising at least one carbon atom.

As disclosed herein, the organic material is a material which is solid at ambient temperature and at atmospheric pressure.

The at least one organic material can thus be chosen from organic polymers and oligomers, whether natural or synthetic.

The polymers and oligomers as disclosed herein can, for example, be obtained by radical polymerization and/or by polycondensation.

The at least one organic material can thus be chosen from poly(vinyl alcohol)s, poly(lactic acid)s, poly(glycolic acid)s, copolymers of lactic acid and of glycolic acid, polystyrenes, poly(methyl(meth)acrylate)s, acrylic and methacrylic copolymers, polyamides, polyesters, polyurethanes and polyureas.

The at least one organic material can also be chosen from cellulose and derivatives thereof, such as alkyl- and hydroxyalkylcelluloses, for example, methylcellulose, ethylcellulose and hydroxyethylcellulose; and cellulose esters, for example, cellulose acetate phthalate.

Finally, the at least one organic material can be chosen from gelatin, pectin, optionally crosslinked, for example, with glutaraldehyde, and polysaccharides, such as carrageenan.

In one embodiment, the at least one organic material is a styrene/methacrylic acid copolymer.

The shell or the capsule comprising at least one organic material may have a thickness ranging from 2 nm to 300 nm, such as from 5 nm to 250 nm, and further such as from 10 nm to 100 nm.

It should be noted that this shell or this capsule, and in accordance with the well-known definition of encapsulation in the technical field, is not a monolayer or a molecular layer but actually a layer which may be described as a “thick” wall, the thickness of which is within the range defined above.

As disclosed herein, the capsule, coating or shell is connected to the core by a physical bond, without covalent bonds. In other words, in the particles incorporated in the compositions as disclosed herein, the core/shell interface is defined as not exhibiting covalent bonds.

The shell or capsule around the metal core in the particles of the compositions as disclosed herein can be formed by various processes.

These processes, which are referred to by the terms of encapsulation or nanoencapsulation process, are known to a person skilled in the art in this technical field and can be divided into two main families: namely, on the one hand, physicochemical processes and, on the other hand, chemical processes.

The physicochemical processes can be chosen from phase separation or coacervation, evaporation, solvent extraction, thermal gelling, controlled precipitation and any other known physicochemical process for microencapsulation.

The chemical processes can be chosen from interfacial polycondensation, interfacial polymerization, polymerization in dispersed medium, in situ polycondensation, emulsion polymerization and any other known chemical process for microencapsulation.

For the physicochemical and chemical processes, reference may also be made to the document “Microencapsulation Methods and Industrial Applications”, (ISBN 0-8247-9703-S).

Among the various encapsulation processes, encapsulation by emulsion polymerization and more specifically to encapsulation by emulsion polymerization as described in the paper “Preparation of Polymer Coated Functionalised Silver Nanoparticles” (J. Am. Chem. Soc., 1999, 121, 10642-10643) can be used.

As is described in the preceding paper, the encapsulation of the nanoparticles is obtained by a conventional radical emulsion polymerization process. However, in order to obtain specific polymerization on the surface of the metal nanoparticles, the micelle phase of the emulsion comprises at least one compound chosen from amphiphilic compounds, the polar part or the hydrophobic part of which having an affinity to the metal surface.

Such a process makes it possible to obtain a core-shell system composed of a metal core and a polymer capsule with a thickness of equal to or greater than 2 nm and conventionally ranging from 2 nm to 300 nm.

Among the various constituents which can be made use of in carrying out the emulsion polymerization described above, examples include:
water and/or ethanol or their mixtures as continuous phase of the emulsion; in one embodiment, water is used;
amphiphilic molecules, the polar part of which comprises at least one carboxyl functional group. Among the amphiphilic molecules, fatty acids such as oleic acid can, for example, be used;
a mixture of styrene and of methacrylic acid as precursor monomers of the capsule; and
silver nanoparticles as support to be encapsulated.

The outer surface of the particles, i.e., the outer surface of the capsule or of the shell, can be covalently modified by at least one chemical group which is capable of improving the adsorption of the particles on keratinous substances, such as the hair. This surface can also be covalently modified by at least one chemical group capable of reacting chemically with keratinous substances, such as the hair.

In the first case, the adsorption on keratinous substances, such as the hair, of the core-shell nanoparticles of the compositions as disclosed herein can be improved by covalently modifying the capsule of organic material, such as a polymer, with various chemical groups (Group A below) which render the surface of the particles, for example, more hydrophobic, more cationic, more anionic and/or more hydrophilic.

The adsorption is defined as employing lower bonding energies than covalent bonds, i.e., less than 50 kcal/mol, between the keratinous substance, such as the individual hair, and the particle. These low-energy bonds are, for example, Van der Waals forces, hydrogen bonds, electron donor-acceptor complexes, and the like.

The group capable of improving the adsorption of the particles on keratinous substances may be chosen from the groups of the following Group A:

Group A:
carboxylic acids and their salts,
primary, secondary, tertiary and quaternary amines,
phosphates,
sulphur oxides, such as sulphones, sulphonic, sulphoxides and sulphates, and
aromatic rings, such as phenyl, triazine, thiophene and imidazole.

In the second case, the adhesion to keratinous substances, such as the hair, of the nanoparticles as disclosed herein is obtained by covalently modifying the capsule of organic material with various groups (Group B) capable of reacting chemically with the keratinous substance. More specifically, the term “groups having a reactivity with regard to the keratinous substance, such as the individual hair,” means the groups capable of forming a covalent bond with this substance, for example, with the amines, carboxylic acids and/or the thiols of the amino acids constituting the keratinous substance. The formation of these covalent bonds can either be spontaneous or can be carried out by activation by temperature, pH, light, a coreactant or a chemical or biochemical catalyst, such as an enzyme.

The group capable of reacting chemically or able to react chemically with keratinous substances, such as the hair, may be chosen from the groups of the following Group B:

Group B:
epoxides,
vinyl and activated vinyl: such as acrylonitrile, acrylic and methacrylic esters, crotonic acid and esters, cinnamic acid and esters, styrene and derivatives, butadiene, vinyl ethers, vinyl ketones, maleic esters, maleimides, vinyl sulphones, and the like,
carboxylic acids and their derivatives: such as anhydride, acid chloride, esters,
acetals, hemiacetals,
aminals, hemiaminals,
ketones and α-hydroxyketones, α-haloketones,
lactones, thiolactones,
isocyanates,
thiocyanates,
imines,
imides (succinimides, glutimides),
pyridyldithio,
N-hydroxysuccinimide esters,
imidates,
oxazine and oxazoline,
oxazinium and oxazolinium,
groups of formula R₁X wherein R₁ is chosen from C₁-C₃₀ alkyl groups, C₆-C₃₀ aryl groups and C₇-C₃₀ aralkyl groups (wherein the alkyl group is chosen from C₁-C₃₀ alkyl groups) and X is a leaving group such as I, Br, Cl, OSO₃R, wherein R is chosen from H and C₁-C₃₀ alkyl groups, —SO₂R′, wherein R′ is chosen from H and C₁-C₃₀ alkyl groups, a tosyl group, N(R″)₃, wherein R″ is chosen from C₁-C₃₀ alkyl groups, and OPO₃R′″₂, wherein R′″ is chosen from H and C₁-C₃₀ alkyl groups; for example, groups of formula R₁X can be chosen from alkyl, aryl and aralkyl halides;
groups of formula R₂X wherein R₂ is chosen from C₃-C₃₀ carbon rings and unsaturated heterocycles with 3 to 20 ring members comprising at least one heteroatom chosen from N, S, O and P, and X is a leaving group as defined above; for example, groups of formula R₂X can be chosen from the halides of unsaturated rings, such as chlorotriazine, chloropyrimidine, chloroquinoxaline and chlorobenzotriazole,
groups of formula R₃SO₂X, wherein R₃ has the same meaning as R₁ and X is a leaving group and has the same meaning given above,
lactones,
thiolactones, and
siloxanes.

Mention may be made, by way of example, of the activation by N-hydroxysulphosuccinimide of core-shell particles of silver/styrene-methacrylic copolymer type. The sulphosuccinimide functional groups are, in the case of an organic shell or capsule, grafted to the surface of the particles via the carboxyl groups which the polymer of the capsule possesses. Such a surface group makes it possible to covalently bond the nanoparticles as disclosed herein to the hair by reaction with the free surface amines which the hair fiber possesses (see reaction scheme below).

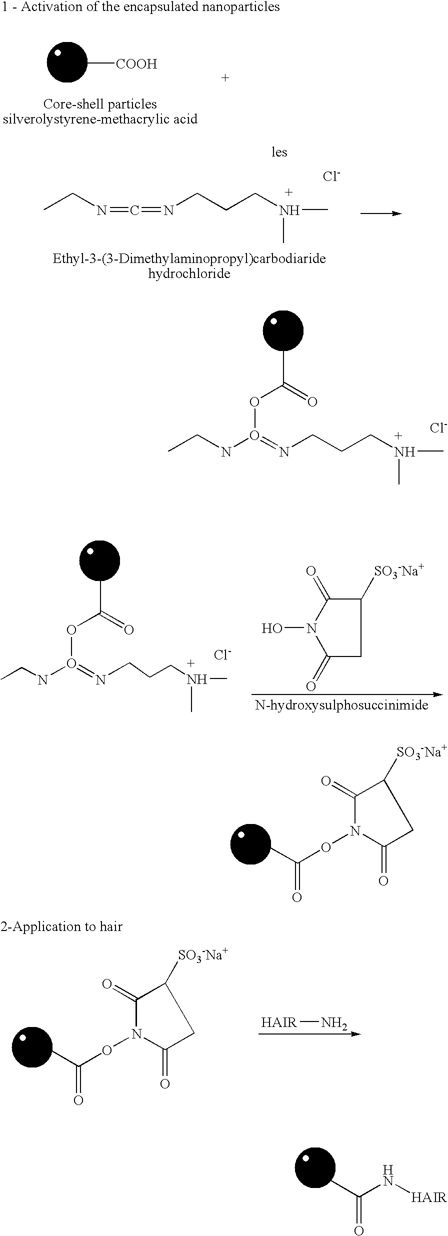

It should be noted that the chemical functional groups on the surface of the keratinous substance, for example, the hair fiber, can be increased in density by pretreatment of the fiber with a solution of polymer having a particular affinity for the fiber and exhibiting reactive functional groups. In the preceding example, the density of the amine functional groups at the surface of the fiber can be increased, for example, by absorbing polyethyleneimine beforehand.

In order to increase the durability of the effect over time, in addition to the improvement in the adhesion or in the adsorption, it is possible to use metal particles encapsulated by a shell of reactive polymer capable of creating interparticle covalent bonds after evaporation of the solvent phase.

In the cosmetic compositions, the encapsulated metal nanoparticles as disclosed herein may be present in a concentration ranging from 0.0001% to 50%, such as from 0.01% to 5% and further such as from 0.05% to 2% by weight of the total weight of the composition.

The composition as disclosed herein may further comprise a physiologically acceptable medium. As disclosed herein the “physiologically acceptable medium” means a medium capable of being applied to keratinous substances or fibers such as the hair of human beings.

The physiologically acceptable medium of the composition may comprise at least one solvent. The solvent makes it possible, for example, to convey the encapsulated metal nanoparticles. The solvent can be chosen from organic solvents, water and their mixtures.

The organic solvents useful herein may be chosen from C₁ to C₄ aliphatic alcohols, such as ethanol and isopropanol, polyols, such as glycerol and propylene glycol, aromatic alcohols, such as benzyl alcohol, alkanes, such as C₅ to C₁₀ alkanes, acetone, methyl ethyl ketone, methyl acetate, butyl acetate, alkyl acetate, dimethoxyethane, diethoxyethane and their mixtures.

The compositions as disclosed herein can be packaged in various forms such as in an aerosol device.

The composition as disclosed herein can further comprise at least one propellant. The propellant comprises the compressed or liquefied gases commonly employed for the preparation of aerosol compositions. For example, air, carbon dioxide gas, compressed nitrogen and a soluble gas, such as dimethyl ether, halogenated (such as fluorinated) and nonhalogenated hydrocarbons, and their mixtures can be used.

The compositions as disclosed herein can further comprise at least one cosmetic additive chosen from conventional cosmetic additives, such as reducing agents, oxidizing agents, thickening agents, softeners, antifoaming agents, direct and oxidation dyes, pearlescent agents, fragrances, peptizing agents, preservatives, and anionic and amphoteric surfactants.

The cosmetic composition as disclosed herein can be a cosmetic treatment composition, such as a composition for contributing sheen to keratinous substances. In one embodiment, it is a hair cosmetic composition, such as a composition for contributing sheen to the hair.

The hair cosmetic compositions as disclosed herein, after application to the hair, can be rinsed out or left in. The compositions, such as hair compositions, (formulations) can be provided in various dosage forms, such as a lotion, a spray, a foam, a lacquer, a conditioner and a shampoo.

The invention will be better understood with the help of the nonlimiting illustrative examples which follow. In the examples, the percentages are expressed by weight and a.m. means active material.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result. The amounts are given as percentages by weight of the total weight of the composition.

### COMPARATIVE EXAMPLE

In this example, a composition according to the present invention and a composition not according to the present invention were prepared.

Composition 1: Aerosol foam according to the invention

| Starch acetate | 5% a.m. |
|---|---|
| Polysorbate 20 | 0.1% a.m. |
| Cocamidopropyl betaine | 0.5% a.m. |
| Encapsulated silver | 1.0% a.m. |
| nanoparticles^{[1]} | |
| Laureth-4 | 0.3% a.m. |
| Isobutane/butane/propane | 5% a.m. |
| Preservative | q.s. |
| Fragrance | q.s. |
| Water | q.s. for 100% |
| Polysorbate 20: Polyoxyethylene (20) sorbitan monolaurate, sold by Atlas. | |
| Laureth-4 surfactant: sold by Uniquema. | |
| Butane/isobutane/propane mixture: 24/56/20. | |
| ^{[1]}Encapsulation was obtained by radical polymerization as an emulsion in water, as is described | |
| # in the paper “Preparation of Polymer Coated Functionalised Silver” (J. Am. Chem. Soc., 1999, 121, 10642-10643). | |
| # The silver nanoparticles used for the encapsulation are sold under the reference “Colloid Mag” by Grant Industries. | |
| # The precursor monomers of the capsule comprises a mixture of styrene and methacrylic acid with a styrene/methacrylic acid molar ratio | |
| # of 40:1. The micelle phase of the emulsion comprises oleic acid. As is shown by the transmission electron microscopy (TEM) exposures | |
| # taken, the particles thus encapsulated exist in the form of a native silver core with a diameter ranging from 5 to 30 nm covered by | |
| # a polymer layer of approximately 5 nm. | |

Composition 2: Control aerosol foam not in
accordance with the invention

| Starch acetate | 5% a.m. |
|---|---|
| Polysorbate 20 | 0.1% a.m. |
| Cocamidopropyl betaine | 0.5% a.m. |
| Nonencapsulated silver | 1.0% a.m. |
| nanoparticles^{[2]} | |
| Laureth-4 | 0.3% a.m. |
| Isobutane/butane/propane | 5% a.m. |
| Preservative | q.s. |
| Fragrance | q.s. |
| Water | q.s. for 100% |
| Polysorbate 20: Polyoxyethylene (20) sorbitan monolaurate, sold by Atlas. | |
| Laureth-4 surfactant: sold by Uniquema. | |
| Butane/isobutane/propane mixture: 24/56/20. | |
| ^{[2]}Silver nanoparticles sold under the reference “Colloid Mag” by Grant Industries. As is shown by the | |
| # transmission electron microscopy (TEM) exposures taken, the nanoparticles exhibit a diameter ranging from 5 nm to 30 nm. | |

Each of the preceding compositions was applied to a lock of brown hair weighing 2.7 g (European hair with a length of 20 cm) at the rate of one gram of composition per lock. After application, the locks were dried under a hairdryer (70° C.) for 30 minutes.

A measurement of sheen was subsequently carried out on a batch of 10 locks treated as indicated above with one or other of the compositions.

The sheen was determined using a photogoniometer by measuring the specular and diffuse reflections of the locks of hair laid flat on a support. Using a 175 watt xenon arc lamp (model ORC175F) coupled to a V filter (lambda), light was emitted over the lock under an angle of +30° C. with respect to the normal to its surface. Using a movable receiving arm, the specular reflection (R), corresponding to the maximum light intensity reflected in the vicinity of an angle of −30°, and the diffuse reflection (D), corresponding to the light reflected at an angle of +15° C., were measured. According to the invention, the sheen was determined by calculating the ratio (R)/(D).

In order to evaluate the retention of the sheen over time, the measurement of sheen was repeated on the same treated locks after storing the treated locks for one month under ambient conditions (20° C. and 50% RH).

The results obtained in terms of stability of the sheen are given in the following Table III:

TABLE III

| | | Sheen | |
|---|---|---|---|
| | Sheen | immediately | Sheen of the |
| | before | after | treated locks after |
| | treatment | treatment | 1 month |
| Composition 1 | 22 ± 2 | 38 ± 2 | 32 ± 4 |
| (in accordance with the | | | |
| invention) | | | |
| Composition 2 | 26 ± 2 | 36 ± 3 | 25 ± 1 |
| (not in accordance with the | | | |
| invention) | | | |

As shown in the preceding Table III, it is recorded that the composition in accordance with the invention retained these sheen properties after storage of the locks under ambient conditions for 1 month. By way of comparison, the composition not in accordance with the invention lost its reflectivity after storage of the locks under ambient conditions for 1 month.

1. A cosmetic composition comprising, in a physiologically acceptable medium,
at least one agent exhibiting a cosmetic activity and
particles comprising a core and a solid shell bonded to the core via a noncovalent bond, wherein the core predominantly comprises at least one metal, the solid shell comprises at least one organic material, and the size of said particles is less than or equal to 500 nm.

2. The composition according to claim 1, wherein the at least one agent exhibiting a cosmetic activity is chosen from:
saccharides, oligosaccharides and polysaccharides which may be optionally hydrolyzed and/or modified,
amino acids, oligopeptides, peptides, proteins, which may be optionally hydrolyzed and/or modified, poly(amino acid)s, and enzymes,
branched and unbranched fatty acids and alcohols,
animal, vegetable, and mineral waxes,
ceramides and pseudoceramides,
hydroxylated organic acids,
UV screening agents,
antioxidants and agents for combating free radicals,
chelating agents,
antidandruff agents,
seborrhoea-regulating agents,
soothing agents,
cationic surfactants,
cationic and amphoteric polymers,
organomodified and nonorganomodified silicones,
mineral, vegetable and animal oils,
polyisobutenes and poly(α-olefin)s,
esters,
soluble and dispersed anionic polymers,
soluble and dispersed non-ionic polymers,
reducing agents,
coloring agents and coloring materials,
foaming agents,
film-forming agents,
particles, other than said particles comprising a core and a solid shell bonded to the core via a noncovalent bond, as defined in claim 1,
and the mixtures thereof.

3. The composition according to claim 2, wherein the coloring agents and coloring materials are chosen from hair dyes.

4. The composition according to claim 1, wherein the at least one agent exhibiting a cosmetic activity is present in a concentration ranging from 0.001% to 10% by weight relative to the total weight of the composition.

5. The composition according to claim 4, wherein the at least one agent exhibiting a cosmetic activity is present in a concentration ranging from 0.01% to 5% by weight relative to the total weight of the composition.

6. The composition according to claim 1, wherein the size of said particles ranges from 1 nm to 500 nm.

7. The composition according to claim 6, wherein the size of said particles ranges from 1 nm to 100 nm.

8. The composition according to claim 7, wherein the size of said particles ranges from 1 nm to 50 nm.

9. The composition according to claim 1, wherein the particles have a shape chosen from spheres, flakes, fibres, tubes, polyhedra and random shapes.

10. The composition according to claim 1, wherein the core of the particles comprises at least 80% by weight of at least one metal.

11. The composition according to claim 10, wherein the core of the particles comprises at least 90% by weight of at least one metal.

12. The composition according to claim 11, wherein the core of the particles comprises 100% by weight of at least one metal.

13. The composition according to claim 1, wherein the at least one metal is chosen from aluminium and all the elements with an atomic number ranging from 21 to 82 and composing Groups 3 to 13 of the Periodic Table of the Elements, and alloys thereof.

14. The composition according to claim 13, wherein the core of the particles comprises a mixture of two or more of said metals and/or alloys thereof.

15. The composition according to claim 1, wherein the core of the particles is a composite core comprising more than one region, with adjacent regions comprising different metals, alloys and/or mixtures thereof.

16. The composition according to claim 15, wherein the composite core of the particles is a multilayer composite core comprising an inner core comprising at least one of metals, alloys and mixtures thereof, at least partially covered by a first layer comprising at least one of metals, metal alloys and mixtures thereof, which is different from that of the inner core and optionally by at least one other layer, wherein each layer at least partially covers the preceding layer and each layer comprises at least one of metals, alloys and mixtures thereof, which is different from the following layer and from the preceding layer.

17. The composition according to claim 1, wherein the core of the particles further comprises at least one stabilizer and unavoidable impurities.

18. The composition according to claim 1, wherein the core of the particles further comprises at least one metal compound other than the at least one metal.

19. The composition according to claim 18, wherein the at least one metal compound is chosen from metal oxides.

20. The composition according to claim 1, wherein the at least one metal is chosen from transition metals, rare earth metals and their alloys and mixtures thereof.

21. The composition according to claim 1, wherein the at least one metal is chosen from aluminium, copper, silver, gold, indium, iron, platinum, nickel, molybdenum, titanium, tungsten, antimony, palladium, zinc, tin and their alloys and mixtures thereof.

22. The composition according to claim 21, wherein the at least one metal is chosen from gold, silver, palladium, platinum and their alloys and mixtures thereof.

23. The composition according to claim 22, wherein the at least one metal is silver.

24. The composition according to claim 1, wherein the solid shell is in direct contact with the at least one metal.

25. The composition according to claim 1, wherein the core is modified at the surface by a treatment which modifies the properties of the core.

26. The composition according to claim 25, wherein said treatment comprises stabilizing the surface of the core by an adsorbed or covalently bonded monolayer.

27. The composition according to claim 1, wherein the at least one organic material is chosen from organic polymers and oligomers.

28. The composition according to claim 27, wherein the at least one organic material is chosen from poly(vinyl alcohol)s, poly(lactic acid)s, poly(glycolic acid)s, copolymers of lactic acid and of glycolic acid, polystyrenes, poly(methyl(meth)acrylate)s, acrylic and methacrylic copolymers, polyamides, polyesters, polyurethanes and polyureas.

29. The composition according to claim 27, wherein the at least one organic material is chosen from cellulose and derivatives thereof.

30. The composition according to claim 29, wherein the at least one organic material is chosen from alkyl- and hydroxyalkylcelluloses and cellulose esters.

31. The composition according to claim 30, wherein the alkyl- and hydroxyalkylcelluloses are chosen from at least one of methylcellulose, ethylcellulose and hydroxyethylcellulose.

32. The composition according to claim 30, wherein the cellulose esters are cellulose acetate phthalate.

33. The composition according to claim 27, wherein the at least one organic material is chosen from gelatin, optionally crosslinked pectin, and polysaccharides.

34. The composition according to claim 33, wherein the pectin is optionally crosslinked with glutaraldehyde.

35. The composition according to claim 33, wherein the polysaccharides are carrageenan.

36. The composition according to claim 28, wherein the at least one organic material is a styrene-methacrylic acid copolymer.

37. The composition according to claim 1, wherein the solid shell of the particles has a thickness ranging from 2 nm to 300 nm.

38. The composition according to claim 37, wherein the solid shell of the particles has a thickness ranging from 5 nm to 250 nm.

39. The composition according to claim 38, wherein the solid shell of the particles has a thickness ranging from 10 nm to 100 nm.

40. The composition according to claim 1, wherein the solid shell is formed by a physicochemical process chosen from phase separation, coacervation, evaporation, solvent extraction, thermal gelling and controlled precipitation.

41. The composition according to claim 1, wherein the solid shell is formed by a chemical process chosen from interfacial polycondensation, interfacial polymerization, polymerization in disperse medium, in situ polycondensation and emulsion polymerization.

42. The composition according to claim 1, wherein the outer surface of the particles is covalently modified by at least one chemical group which is capable of improving the adsorption of the particles on keratinous substances.

43. The composition according to claim 42, wherein the keratinous substance is hair.

44. The composition according to claim 1, wherein the outer surface of the particles is covalently modified by at least one chemical group capable of reacting chemically with keratinous substances.

45. The composition according to claim 44, wherein the keratinous substance is hair.

46. The composition according to claim 42, wherein the at least one chemical group capable of improving the adsorption of the particles on keratinous substances is chosen from the following groups:
carboxylic acids and salts thereof,
primary, secondary, tertiary and quaternary amines,
phosphates,
sulphur oxides, and
aromatic rings.

47. The composition according to claim 46, wherein the sulphur oxides are chosen from sulphones, sulphonic, sulphoxides and sulphates.

48. The composition according to claim 46, wherein the aromatic rings are chosen from phenyl, triazine, thiophene and imidazole.

49. The composition according to claim 44, wherein the at least one chemical group capable of reacting chemically with keratinous substances is chosen from the following groups:
epoxides,
vinyl and activated vinyl,
carboxylic acids and derivatives thereof,
acetals, hemiacetals,
aminals, hemiaminals,
ketones and α-hydroxyketones, α-haloketones,
lactones, thiolactones,
isocyanates,
thiocyanates,
imines,
imides,
pyridyldithio,
N-hydroxysuccinimide esters,
imidates,
oxazine and oxazoline,
oxazinium and oxazolinium,
groups of formula R₁X wherein R₁ is chosen from C₁-C₃₀ alkyl groups, C₆-C₃₀ aryl groups, and C₇-C₃₀ aralkyl groups with the alkyl group chosen from C₁-C₃₀ alkyl groups; and X is a leaving group,
groups of formula R₂X wherein R₂ is chosen from C₃-C₃₀ carbon rings and unsaturated heterocycles with 3 to 20 ring members comprising at least one heteroatom chosen from N, S, O and P; and X is a leaving group,
groups of formula R₃SO₂X, wherein R₃ is chosen from C₁-C₃₀ alkyl groups, C₆-C₃₀ aryl groups, and C₇-C₃₀ aralkyl groups with the alkyl group chosen from C₁-C₃₀ alkyl groups; and X is a leaving group,
lactones,
thiolactones, and
siloxanes.

50. The composition according to claim 49, wherein the vinyl and activated vinyl groups are chosen from acrylonitrile, acrylic and methacrylic esters, crotonic acid and esters, cinnamic acid and esters, styrene and derivatives, butadiene, vinyl ethers, vinyl ketones, maleic esters, maleimides, and vinyl sulphones.

51. The composition according to claim 49, wherein the carboxylic acids and derivatives thereof are chosen from anhydrides, acid chlorides, and esters.

52. The composition according to claim 49, wherein the imides are chosen from succinimides and glutimides.

53. The composition according to claim 49, wherein the leaving group in the groups of the formulae R₁X, R₂X, and R₃SO₂X is chosen from I, Br, Cl, OSO₃R, wherein R is chosen from H and C₁-C₃₀ alkyl groups, —SO₂R′, wherein R′ is chosen from H and C₁-C₃₀ alkyl groups, a tosyl group, N(R″)₃, wherein R″ is chosen from C₁-C₃₀ alkyl groups, and OPO₃R′″₂, wherein R′″ is chosen from H and C₁-C₃₀ alkyl groups.

54. The composition according to claim 49, wherein the groups of the formula R₁X are chosen from alkyl, aryl and aralkyl halides.

55. The composition according to claim 49, wherein the groups of the formula R₂X are chosen from chlorotriazine, chloropyrimidine, chloroquinoxaline and chlorobenzotriazole.

56. The composition according to claim 1, wherein the solid shell of the particles comprises at least one reactive polymer capable of creating interparticle covalent bonds.

57. The composition according to claim 1, wherein the particles are present in a concentration ranging from 0.0001% to 50% by weight relative to the total weight of the composition.

58. The composition according to claim 57, wherein the particles are present in a concentration ranging from 0.01% to 5% by weight relative to the total weight of the composition.

59. The composition according to claim 58, wherein the particles are present in a concentration ranging from 0.05% to 2% by weight relative to the total weight of the composition.

60. The composition according to claim 1, wherein the physiologically acceptable medium comprises at least one solvent.

61. The composition according to claim 60, wherein the at least one solvent is chosen from organic solvents, water and the mixtures thereof.

62. The composition according to claim 61, wherein the organic solvents are chosen from C₁ to C₄ aliphatic alcohols, polyols, aromatic alcohols, alkanes, acetone, methyl ethyl ketone, methyl acetate, butyl acetate, alkyl acetate, dimethoxyethane, diethoxyethane and the mixtures thereof.

63. The composition according to claim 62, wherein the C₁ to C₄ aliphatic alcohols are chosen from ethanol and isopropanol.

64. The composition according to claim 62, wherein the polyols are chosen from glycerol and propylene glycol.

65. The composition according to claim 62, wherein the aromatic alcohols are benzyl alcohol.

66. The composition according to claim 62, wherein the alkanes are chosen from C₅ to C₁₀ alkanes.

67. The composition according to claim 1, further comprising at least one cosmetic additive chosen from reducing agents, oxidizing agents, thickening agents, softeners, antifoaming agents, direct and oxidation dyes, pearlescent agents, fragrances, peptizing agents, preservatives, and anionic and amphoteric surfactants.

68. The composition according to claim 1, wherein the composition is chosen from cosmetic compositions which contribute sheen to keratinous substances.

69. The composition according to claim 68, wherein the composition is a hair composition for contributing sheen to hair.

70. The composition according to claim 69, wherein the hair composition is provided in a form chosen from the forms of a lotion, a spray, a foam, a lacquer, a conditioner and a shampoo.

71. The composition according to claim 1, wherein the composition is packaged in an aerosol device.

72. A cosmetic process for the treatment of keratinous substances, comprising applying to the keratinous substances a composition comprising, in a physiologically acceptable medium,
at least one agent exhibiting a cosmetic activity and
particles comprising a core and a solid shell bonded to the core via a noncovalent bond, wherein the core predominantly comprises at least one metal, the solid shell comprises at least one organic material, and the size of said particles is less than or equal to 500 nm.

73. The process according to claim 72, wherein the keratinous substance is hair.

74. The process according to claim 72, wherein the process is for contributing sheen to the keratinous substances.

75. The process according to claim 74, wherein the keratinous substance is hair.

76. A process of making a cosmetic composition, comprising
adding to a composition particles comprising a core and a solid shell bonded to the core via a noncovalent bond, wherein the core predominantly comprises at least one metal, the solid shell comprises at least one organic material, and the size of said particles is less than or equal to 500 nm,
wherein the composition comprises, in a physiologically acceptable medium, at least one agent exhibiting a cosmetic activity and the resulting cosmetic composition is for contributing sheen to keratinous substances.

77. The process according to claim 76, wherein the keratinous substance is hair.
